(19) United States
(12) Patent Application Publication
(10) Pub. No.: US 2008/0039883 A1
(43) Pub. Date: Feb. 14, 2008

(54) Anti-coring device for a surgical morcellator

(76) Inventor: Martin J. Nohilly, Murray Hill, NJ (US)

Correspondence Address: BODNER & O'ROURKE, LLP, 425 BROADHOLLOW ROAD, SUITE 108, MELVILLE, NY, 11747, US

(21) Appl. No.: 11/502,339

(22) Filed: Aug. 10, 2006

(51) Int. Cl. A61B 17/32

(52) U.S. Cl. 606/180; 600/567

(57) ABSTRACT

An anti-coring device for a surgical morcellator, which morcellator has a rotatable cutting blade having a sharpened edge and an outer sleeve that is axially moveable on the cutting blade, includes a shield mounted on the distal end of the outer sleeve and axially moveable therewith to selectively cover and at least partially uncover the sharpened edge of the rotatable cutting blade. The shield includes a main body and a protrusion extending axially from the main body and partially about the circumference of the cutting blade. The shield is axially positionable on the cutting blade so that is selectively covers the entire circumference of the sharpened edge of the cutting blade with its main body or only covers a portion of the circumference of the sharpened edge of the cutting blade with its protrusion, leaving the remaining portion of the sharpened edge exposed.

### BACKGROUND OF THE INVENTION

1. Field of the Invention

The present invention relates generally to surgical devices and methods, and more particularly to a laparoscopic morcellator and methods of using the morcellator during a surgical procedure.

2. Description of the Prior Art

Minimally invasive surgical procedures, such as laparoscopic procedures, have become very common. These procedures typically involve one or more small incisions that provide access to the relevant internal organ or tissue. A trocar, cannula or the like is placed into each incision, and all surgical steps are subsequently performed with instruments passed through or into the trocar(s).

Many times it is desirable to remove relatively large masses of tissue, for example a uterine fibroid, which can be difficult and time consuming given the diameter of the trocar. To this end, laparoscopic morcellators have been developed to assist in severing the tissue mass into pieces that can readily be removed through the trocar. An example of one such a morcellator is described in detail in U.S. Pat. No. 6,039,748, which issued to George M. Savage, et al., the disclosure of which is incorporated herein by reference in its entirety.

Known morcellators typically include a rotating tube having a sharp distal cutting edge, which rotates within an outer stationary tube. The morcellator is inserted through a cannula or trocar, or more commonly directly through the incision. A grasping instrument (i.e., tenaculum) is inserted through the inner rotating tube. Using the tenaculum, the surgeon pulls the tissue to be severed up into the tube so that the rotating edge of the inner tube severs the grasped portion of tissue. By repeating the grasping and severing procedure, the surgeon can remove the large tissue mass in increments.

Another technique surgeons have developed to improve the speed of tissue removal using a morcellator is known as “orange peeling.” In orange peeling, the cylindrical blade of the morcellator is held on a plane with the outside of the organ or tissue being removed in such a way as to allow the organ or tissue to be rotated. This allows a longer strip to be removed as opposed to the “coring” technique described above, which limits the length of the strip removed to the thickness of the organ. Orange peeling requires skill of the surgeon holding the morcellator as well as skill of the assistant that is passing tissue to the morcellator with a second grasper in the cavity. The skill required is in keeping the blade at the surface of the tissue without either allowing the blade to dive in, or “core”, and at the same time not leaving the surface so much that the tissue strip becomes thin or breaks. Orange peeling is better from a safety standpoint as well, as the blade remains visible at all times to the user. Thus, it would be desirable to provide a morcellator having improved feature(s) that facilitate the ability of the surgeon to use the orange peeling technique.

Another difficulty sometimes encountered with known morcellators is that during use, whether by coring or orange peeling, the amount of tissue being withdrawn can cause friction within the inner rotating tube or to the seal system during removal. The larger the tissue sections or strips, the more exaggerated this problem becomes. It would further be desirable to provide a morcellator that lowers such withdrawal forces.

In addition to friction encountered during tissue removal, manipulation of the grasping instrument within the rotating inner tube can interfere with the blade rotation and tends to lead to dulling of the blade with known morcellators, since the sharp edge is positioned on the inner most point on the circumference of the inner tube. It would also be desirable to provide a morcellator that provides increased protection against such interference and blade dulling.

Finally, as indicated above, morcellators are typically inserted through a cannula, or more commonly directly through the incision. When inserted directly into the incision the existing trocar must first be removed. Following morcellation, if any other procedures or tasks are to be performed within the cavity, the morcellator must be removed before any other laparoscopic instrument can be inserted through that same portal. Removal and reinsertion of trocars and laparoscopic instruments during a given procedure is awkward and time consuming, and creates additional trauma at the site. It is further desirable to provide a morcellator that will greatly reduce the need for such exchanges.

### OJBECTS AND SUMMARY OF THE INVENTION

It is an object of the present to provide a device for use on a surgical morcellator that prevents the cutting blade of the morcellator from coring into an anatomical body of a patient being laparoscopically removed.

It is another object of the present invention to provide a surgical morcellator that facilitates the removal of tissue from a patient during a surgical procedure through the use of a commonly used and preferred technique known as “orange peeling”.

It is still another object of the present invention to provide an anti-coring device for a surgical morcellator which facilitates the removal of larger and/or longer transected tissue morsels during a surgical procedure.

It is a further object of the present invention to provide a surgical morcellator which requires less skill to operate.

It is yet a further object of the present invention to provide an anti-coring device for a surgical morcellator which enhances safety by providing constant visualization of the morcellator cutting blade and the location of the cutting blade with respect to an anatomical body being removed during a laparoscopic procedure.

It is yet a further object of the present invention to provide an anti-coring device for a surgical morcellator which maintains the preferred maximum circumference of the sharpened edge of the morcellator cutting blade that is in contact with a tissue organ being removed during a surgical procedure in which the “orange peeling” technique is being used.

It is still a further object of the present invention to provide a method for transecting tissue using a surgical morcellator having an anti-coring device formed in accordance with the present invention.

In accordance with one form of the present invention, an anti-coring device for a surgical morcellator in which the surgical morcellator has a rotatable cylindrical cutting blade having a distal end and a sharpened edge situated at the distal end includes a shield situated on the distal end of the cutting blade and axially moveable thereon. The shield includes a main body having a bore formed axially therethrough for receiving a portion of the cutting blade, and a protrusion extending axially from the main body and partially about the circumference of the cutting blade. The shield is axially positionable on the cutting blade in a first position in which the main body thereof is disposed axially in alignment with the sharpened edge of the rotatable cutting blade to cover the entire circumference of the sharpened edge of the cutting blade. The shield is also axially positionable on the cutting blade in at least a second position in which the protrusion is disposed axially in alignment with the sharpened edge of the rotatable cutting blade to cover a selected arcuate first portion of the circumference thereof and to expose a second portion of the circumference of the sharpened edge of rotatable cutting blade.

In accordance with another form of the present invention, a method of laparoscopically removing an anatomical body from a patient during a surgical procedure includes the step of using a surgical morcellator having an anti-coring device as described previously. The surgical morcellator includes an outer sleeve having a bore formed axially therethrough for receiving at least a portion of the rotatable cutting blade. The outer sleeve further has a distal end situated in proximity to the distal end of the cutting blade, and being axially moveable on the rotatable cutting blade. The shield of the present invention, such as described previously, is mounted on the distal end of the outer sleeve and axially moveable therewith to selectively cover and at least partially uncover the sharpened edge of the rotatable cutting blade.

The method of laparoscopically removing an anatomical body from a patient during a surgical procedure further includes the steps of positioning the shield of the anti-coring device in a first position on the rotating cutting blade in which the entire circumference of the sharpened edge of the cutting blade is covered; inserting the distal end of the outer sleeve of the surgical morcellator into a patient; positioning the shield of the anti-coring device in at least a second position with respect to the rotatable cutting blade in which a selected arcuate first portion of the circumference of the sharpened edge of the blade is covered and a second portion of the circumference of the sharpened edge of the cutting blade is exposed; and engaging the second portion of the sharpened edge of the morcellator cutting blade exposed by the shield when the shield is in the at least second position with the anatomical body for transecting tissue therefrom and for the removal of the body from the patient.

These and other objects, features and advantages of the present invention will be apparent from the following detailed description of illustrative embodiments thereof, which is to be read in connection with the accompanying drawings.

### BRIEF DESCRIPTION OF THE DRAWINGS

FIG. 1 is an enlarged perspective view of the distal end portion of a conventional morcellator.

FIG. 2 is a side view of a morcellator incorporating an anti-coring device formed in accordance with the present invention.

FIG. 3 is a cross-sectional view of the distal end portion of the morcellator and the anti-coring device of the present invention situated thereon, with the anti-coring device being in a second position.

FIG. 4 is an enlarged perspective view of the distal end portion of the morcellator with the anti-coring device of the present invention situated thereon, the anti-coring device being shown in the second position.

FIG. 5 is an enlarged perspective view, taken from a different angle from that shown in FIG. 4, and the anti-coring device of the present invention situated thereon, the anti-coring device being shown in the second position.

FIG. 6 is an enlarged cross-sectional view of the distal end portion of the morcellator and the anti-coring device of the present invention situated thereon, the anti-coring device being shown in the second position.

FIG. 7 is an enlarged side view of the distal end portion of the morcellator and the anti-coring device of the present invention situated thereon, the anti-coring device being shown in the second position.

FIG. 8 is an enlarged cross-sectional view of the distal end portion of the morcellator and the anti-coring device of the present invention situated thereon, the anti-coring device being shown in a first position.

FIG. 9 is an enlarged side view of the distal end portion of the morcellator and the anti-coring device of the present invention situated thereon, the anti-coring device being shown in the first position.

FIG. 10 is a cross-sectional view of the distal end portion of the morcellator and the anti-coring device of the present invention situated thereon, the anti-coring device being shown in a third position.

FIG. 11 is a an enlarged perspective view of the distal end portion of the morcellator and the anti-coring device of the present invention situated thereon, the anti-coring device being shown in the third position.

FIG. 12 is an enlarged side view of the distal end portion of the morcellator and the anti-coring device of the present invention situated thereon, the anti-coring device being shown in the third position.

FIG. 13 is a perspective view of the distal end portion of the morcellator having an anti-coring device formed in accordance with the present invention integrally formed thereon, the anti-coring device being shown in the second position.

FIG. 14 is a perspective view of the distal end portion of the morcellator and the anti-coring device of the present invention situated thereon, the anti-coring device being constructed in accordance with an alternative form of the present invention and being shown in the second position.

FIG. 15 is a perspective view of the distal end portion of the morcellator and the anti-coring device of the present invention situated thereon, shown transecting tissue from an anatomical body of a patient during a laparoscopic surgical procedure using the preferred surgical technique commonly referred to as “orange peeling”.

### DETAILED DESCRIPTION OF THE PREFERRED EMBODIMENTS

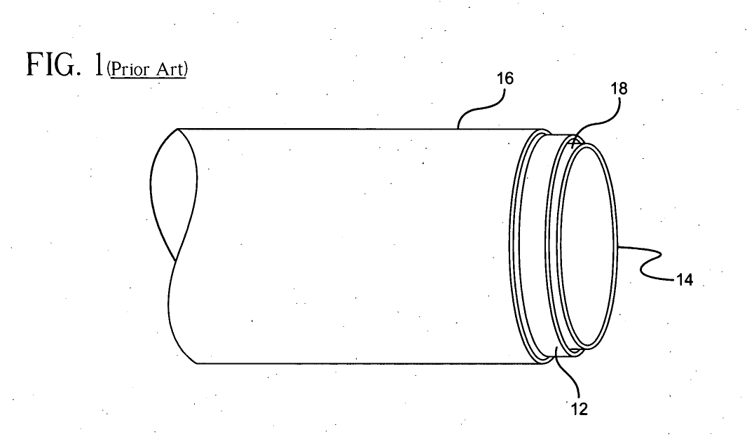

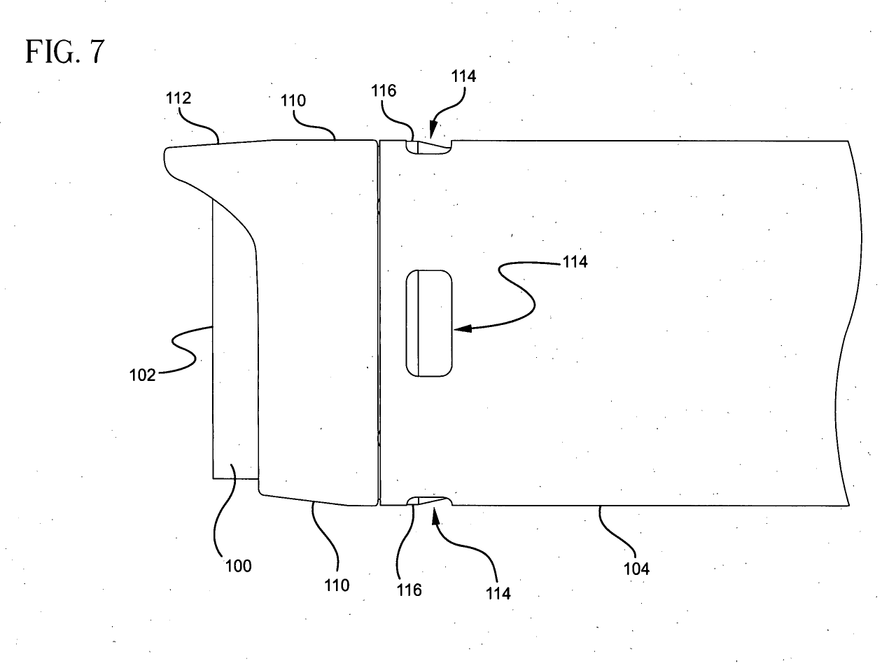

Referring initially to FIG. 1 of the drawings which is a reproduction of FIG. 7C of the aforementioned U.S. Pat. No. 6,039,748 (Savage et al.), it will be seen that a conventional surgical morcellator includes a non-moveable cylindrical outer sleeve 16, the distal end of which is shown in FIG. 1 of the drawings. Outer sleeve 16 includes a bore formed axially therethrough for receiving therein a rotatable cylindrical morcellator cutting blade 12, the distal end of which is also shown in FIG. 1. The cutting blade of the morcellator includes a sharpened edge 18 for transecting the tissue of an anatomical body (i.e., an organ, such as the uterus) of a patient during a laparoscopic surgical procedure to form transected tissue morsels that are withdrawn through the morcellator by using a grasping instrument, such as a tenaculum, as described more fully in the aforementioned Savage et al. patent. The rotatable cutting blade has similarly formed therein an axial bore. As is well known in the art, and as more fully described in the aforementioned Savage et al. patent, the cutting blade is operatively linked to a drive motor (not shown) for rotating the cutting blade within outer sleeve 16.

The conventional surgical morcellator further includes a cylindrical inner sleeve 14, also having an axial bore. Inner sleeve 14 is received by the axial bore of rotating cutting blade 12. Tissue morsels cut from an anatomical body are pulled through the axial bore of inner sleeve 14 by the tissue grasping instrument, or tenaculum.

As is described in the aforementioned Savage et al. patent, inner sleeve 14 is axially moveable with respect to the sharpened edge 18 of cutting blade 12 and, accordingly, acts as a blade guard which protects the blade from inadvertent contact with other surgical instruments and which also prevents inadvertent cutting of tissues during positioning and movement of the laparoscopic surgical morcellator. A sliding guard actuator 94 (not shown in FIG. 1 hereof but shown in FIG. 7B of the aforementioned Savage et al. patent), is operatively linked to inner sleeve 14 to move the inner sleeve with respect to the cutting blade 12.

Inner sleeve 14 of the conventional surgical morcellator may be advanced distally to a first position in which it extends beyond the periphery of the sharpened edge 18 of cutting blade 12, and may be withdrawn axially within rotating cutting blade 12 to a second position in order to expose the full circumference of sharpened edge 18 during the laparoscopic surgical procedure. Inner sleeve 14 in the conventional morcellator shown in the Savage et al. patent does not prevent unintentional coring into the anatomical body being removed when the preferred “orange peeling” technique is being employed.

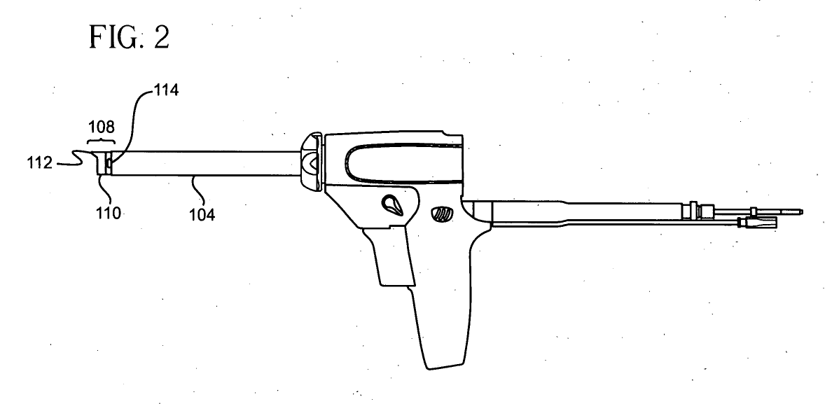

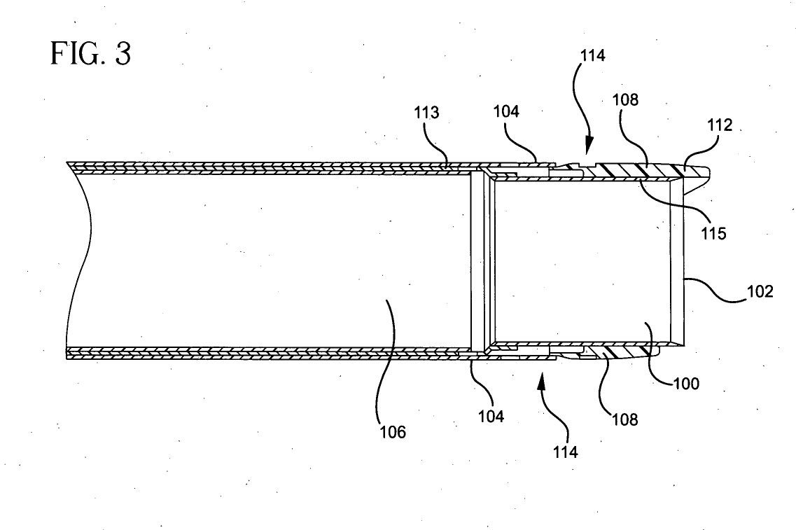

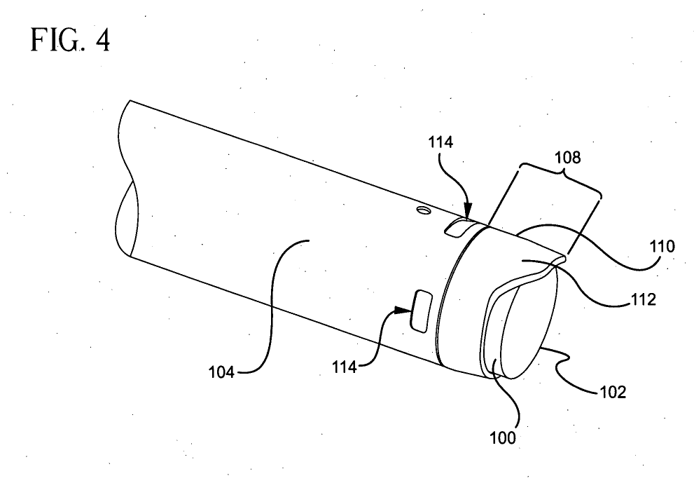

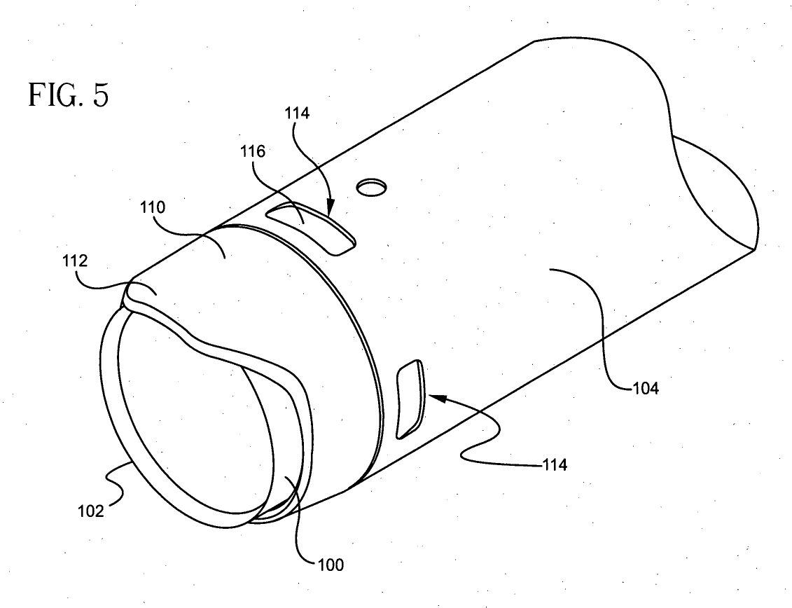

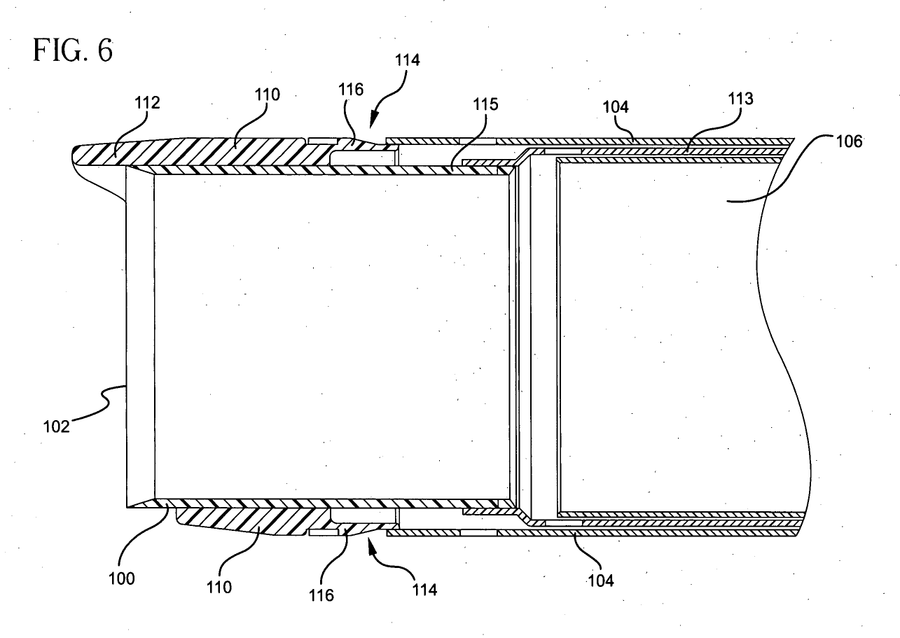

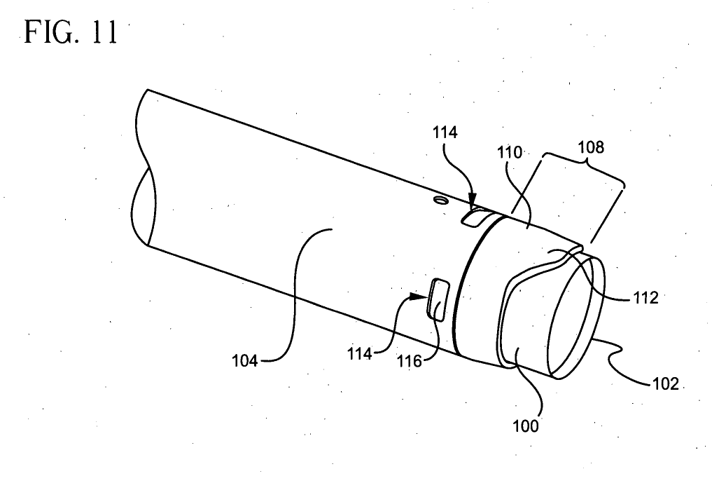

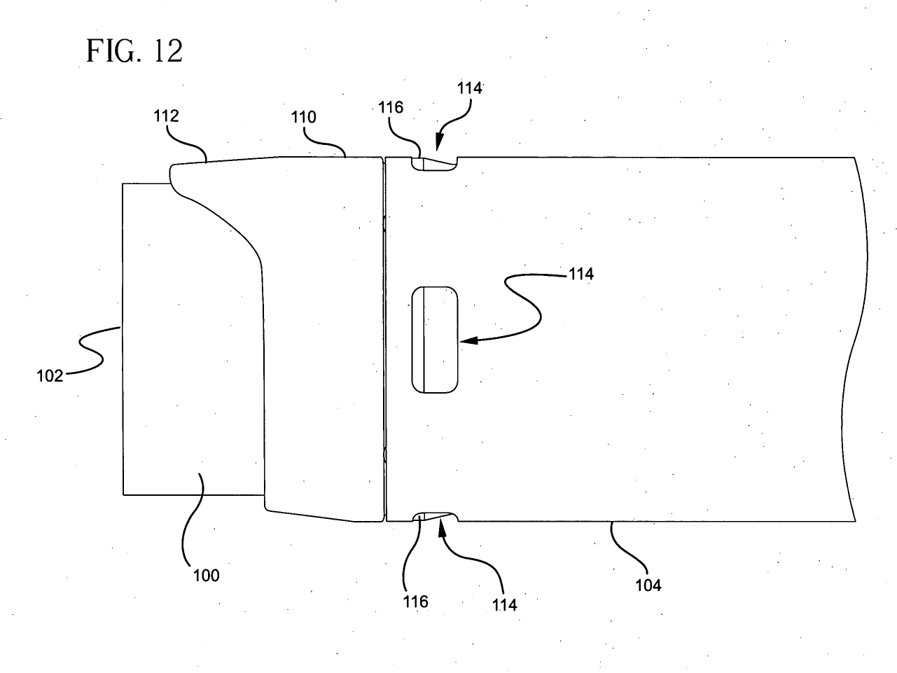

FIG. 2 of the drawings illustrates a surgical morcellator having an anti-coring device constructed in accordance with the present invention. FIGS. 3-15 illustrate in greater detail the distal end portion of the surgical morcellator and, in particular, the anti-coring device of the present invention. The surgical morcellator includes a rotatable cylindrical cutting blade 100 having a distal end and a sharpened edge 102 situated at the distal end. The cutting blade 100 may be formed from any number of suitable materials, such as surgical stainless steel, for example, 300 or 400 series medical grade stainless steel, which is known to retain a sharpened edge and which will not corrode. The cutting blade 100 is operatively linked to a drive mechanism or motor, such as shown and described in the aforementioned Savage et al. patent, in order to rotate the cutting blade 100. The cutting blade 100 had formed therein an axial bore, and may be formed in two sections—a first elongated main section 113 that is driven by a motor, motor linkage or other mechanism (not shown) to rotate, and a shorter tip portion 115 having a diameter which is less than that of the main section 113 and which is joined to the main section by soldering, brazing, adhesively joining the two sections or other ways known in the art.

The surgical morcellator further preferably includes a cylindrical outer sleeve 104 having a bore formed axially therethrough for receiving at least a portion of the rotatable cutting blade 100. The outer sleeve 104 has a distal end situated in proximity to the distal end of the cutting blade 100, and is preferably axially moveable on the rotatable cutting blade 100. The outer sleeve 104 is also preferably made from stainless steel, such as 300 series medical grade stainless steel, but may also be made from other materials such as polyethylene or fiberglass.

The surgical morcellator may also include an inner sleeve 106 which is received within the axial bore of the rotatable cutting blade 100 so that the rotatable cutting blade 100 is disposed between the inner sleeve 106 and the outer sleeve 104 of the morcellator. The inner sleeve 106 also has a bore formed axially therethrough. The bore is provided for passing therethrough tissue morsels transected from an anatomical body of a patient during a laparoscopic surgical procedure, and grasped and pulled through the bore by a tissue grasping instrument, such as a tenaculum. The inner sleeve 106 may also be formed from stainless steel, such as 300 series medical grade stainless steel, or, like the outer sleeve 104, may be formed from a polyethylene or fiberglass material. Preferably the inside surface of the inner sleeve 106 may be made lubricious either in the selection of material used for the inner sleeve 106 or by coating the inside surface of the inner sleeve 106 with a hydrophilic or other coating to reduce friction between the inside surface of the inner sleeve 106 and tissue morsels being withdrawn by the tenaculum therethrough.

It should be noted here that it is envisioned to be within the scope of the present invention to construct the surgical morcellator without an inner sleeve 106 so that the tissue morsels are grasped and withdrawn through the axial bore of the rotatable cutting blade 100.

In accordance with one form of the present invention, and as shown in FIGS. 2-15 of the drawings, a surgical morcellator includes an anti-coring device. The anti-coring device includes a shield 108 mounted on or situated at the distal end of the outer sleeve 104 and axially moveable therewith to selectively cover and at least partially uncover the sharpened edge 102 of the rotatable cutting blade 100.

More specifically, the shield 108 is situated at the distal end of the cutting blade 100 and axially moveable with respect thereto. The shield 108 includes a main body 110 having a bore formed axially therethrough for receiving a portion of the cutting blade 100, and a protrusion 112 or “tooth” extending axially from the main body 110 and partially about the circumference of the cutting blade 100. The shield 108 is axially moveable with the outer sleeve 104 to selectively cover and at least partially uncover the sharpened edge 102 of the rotatable cutting blade 100.

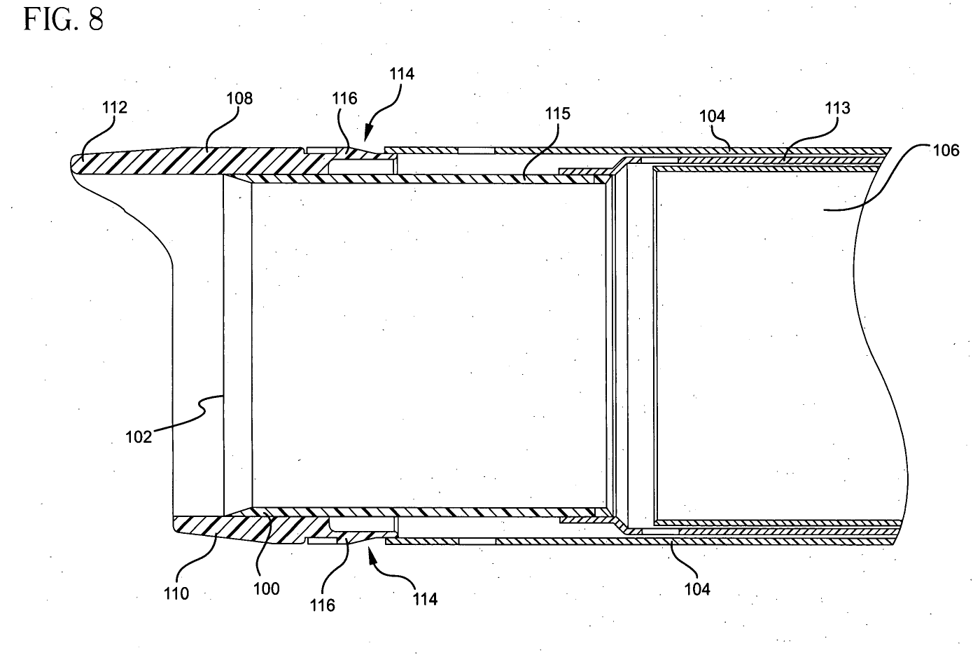

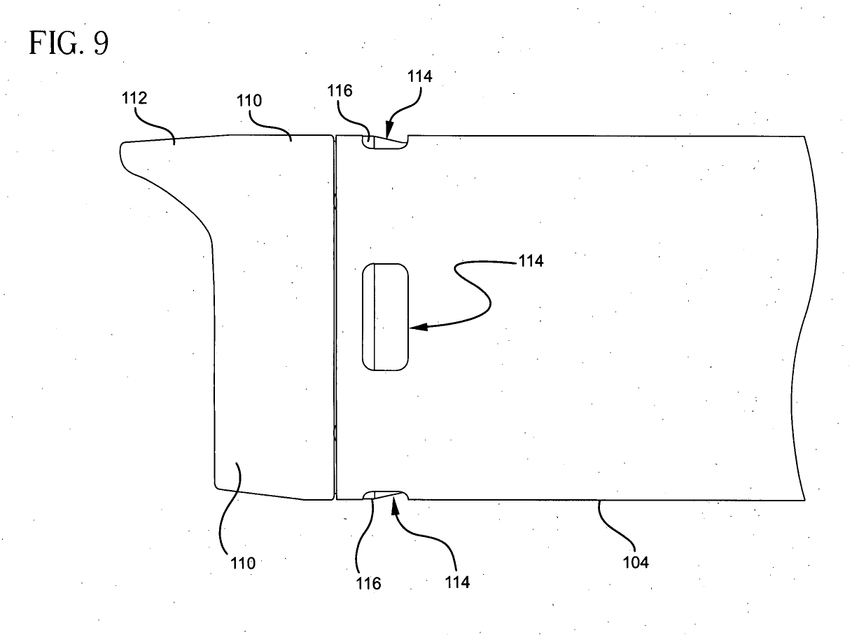

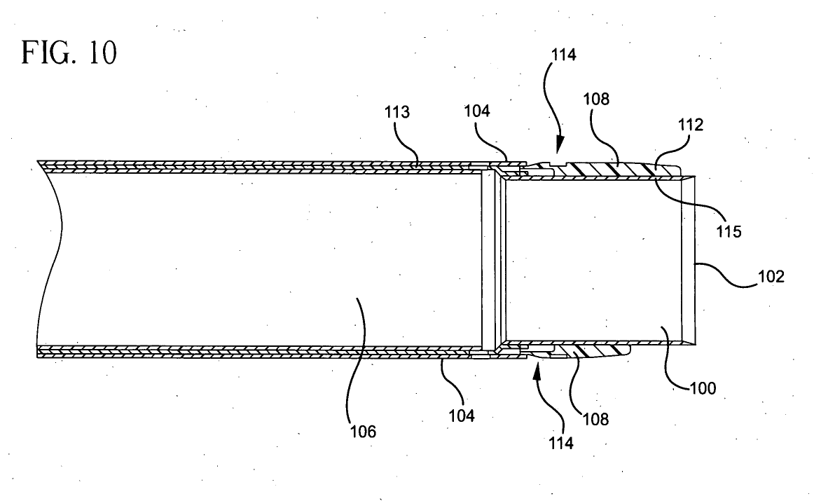

Even more specifically, the shield 108 is axially positionable with respect to the cutting blade 100 in a first position (shown in FIGS. 8 and 9) in which the main body 110 thereof is disposed axially in alignment with the sharpened edge 102 of the rotatable cutting blade 100 to cover the entire circumference of the sharpened edge 102, and at least a second position (shown in FIGS. 3-7 and 13-15) in which the protrusion 112 is disposed axially in alignment with the sharpened edge 102 of the rotatable cutting blade 100 to cover a selected arcuate first portion of the circumference thereof and to expose and not cover a second portion of the circumference of the sharpened edge 102 of the rotatable cutting blade 100. Even more preferably, the shield 108 is axially positionable with respect to the cutting blade 100 in a third position (shown in FIGS. 10-12) in which the shield 108 is in non-alignment with the sharpened edge 102 of the rotatable cutting blade 100 to expose the entire circumference of the sharpened edge 102.

The selected arcuate first portion of the circumference of the sharpened edge 102 of the rotatable cutting blade 100 covered by the protrusion 112, or “tooth,” is preferably between about ninety degrees (90°) and about one hundred twenty degrees (120°) when the shield 108 is in the second position. However, it is preferred that the selected arcuate first portion of the circumference of the sharpened edge 102 of the rotatable cutting blade 100 that is covered by the protrusion 112 is at least about fifty-four degrees (54°) when the shield 108 is in the second position. Stated in another way, it is preferred if about two-thirds (⅔) to about three-quarters (¾) of the circumference of the sharpened edge 102 of the cutting blade 100 is exposed, and at least about fifteen percent (15%) of the sharpened edge 102 is covered, for efficient tissue morcellation using the preferred “orange peeling” technique while providing enough resistance to coring to promote the cutting blade 100 sliding along the surface of the organ being morcellated. If the arcuate extent of the protrusion 112 or “tooth” is too small, that is, somewhat less than fifteen degrees (15°) or about fifty-four percent (54%) in its coverage of the sharpened edge 102 of the rotatable cutting blade 100, the protrusion 112 may not be blunt enough to prevent the protrusion 112 from digging into the tissue, and this may prevent the surgeon from efficiently using the “orange peeling” surgical technique.

It is also preferred that at least a portion of the protrusion 112 of the shield 108 extends at least about 0.030 inches, but more preferably between about 0.070 inches and about 0.100 inches, axially beyond the sharpened edge 102 of the rotatable cutting blade 100, when the shield 108 is in the second position. If the protrusion 112 extends too far beyond the sharpened edge 102 of the cutting blade 100, it may prevent the cutting blade 100 from taking a full “bite” out of the organ when the morcellator is at a steep angle to the tissue being transected. If the protrusion 112 does not extend sufficiently beyond the sharpened edge 102 of the rotatable cutting blade 100, it is possible that the shield 108 will not prevent coring of the organ during the “orange peeling” procedure.

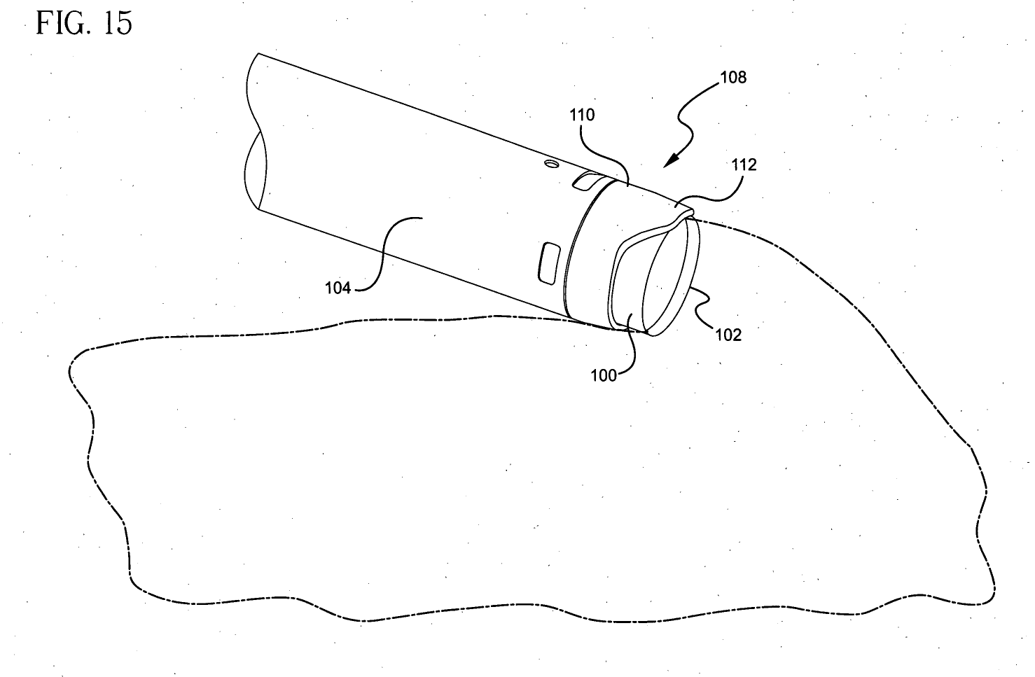

The surgical morcellator of the present invention and, in particular, the anti-coring device used thereon, allow faster, more controlled and safer morcellation of anatomical bodies during a laparoscopic surgical procedure by facilitating the “orange peeling” technique, as shown in FIG. 15 of the drawings. More specifically, during morcellation, one of the techniques clinicians like to use is “orange peeling”, in which the cylindrical cutting blade 100 of the morcellator is held on a plane or at an acute angle with the outside of the organ being morcellated in such a way as to allow the organ to be rotated. This allows a longer strip of tissue to be removed, as opposed to “coring”, which limits the length of the removed tissue strip to the thickness of the organ. Orange peeling as a technique requires skill for the clinician holding the morcellator and for his or her assistant passing the tissue to the morcellator with a second grasping instrument, or tenaculum, in the patient's body cavity. The surgeon must be skilled in keeping the cutting blade 100 of the morcellator at the surface of the tissue without allowing the cutting blade 100 to dive in, or “core”, and at the same time not leaving the surface of the organ so much that the tissue strip becomes thin and breaks. Orange peeling is desirable from a safety standpoint as well, as the cutting blade 100 remains visible at all times to the user.

With the anti-coring device of the present invention fitted on a surgical morcellator, a full “bite” of tissue may be taken while maintaining the device along the surface of the organ without the sensitivity, aim or skill required by conventional surgical morcellators, as a morcellator having the anti-coring device of the present invention can maintain its engagement along the surface of the organ with a much greater range in the angle at which the morcellator is held to the organ surface during the surgical procedure. The morcellator, having the anti-coring device of the present invention mounted thereon, further enables the tissue to be compressed as it is cut, leading to thicker transected tissue strips and faster morcellation procedures.

The shield 108 of the anti-coring device of the present invention may be locked in preferably three positions. As stated previously, the shield 108 in one position (shown in FIGS. 8 and 9) would cover the entire circumference of the sharpened edge 102 of the cutting blade 100 to protect the blade 100 from inadvertent contact with other surgical instruments and to prevent the inadvertent cutting of tissues during positioning and movement of the morcellator. In an intermediate position (shown in FIGS. 3-7 and 13-15), the shield 108 exposes preferably about two-thirds to about three-quarters of the circumference of the sharpened edge 102 of the cutting blade 100, with preferably about one-quarter to about one-third of the circumference of the sharpened edge 102 covered by the shield's 108 protrusion 112 or “tooth”, in order to effect proper “orange peeling”, that is, to cause the cutting blade 100 edge to remain at the surface of the organ being morcellated without “coring” into the organ. In the third position (shown in FIGS. 10-12), the shield 108 is withdrawn axially on the cutting blade 100 to expose the entire circumference of the sharpened edge 102 when techniques other than “orange peeling” are used by the surgeon during the laparoscopic procedure.

The shield 108 may be formed from stainless steel or other material, such as a polymer (e.g., polyethylene) or fiberglass, and may be mounted on the distal end of the outer sleeve 104. Alternatively, the shield 108 may be integrally formed with the outer sleeve 104 at the distal end thereof. In the former situation, the distal end of the outer sleeve 104 may include a plurality of slots 114 formed through the thickness thereof and spaced apart from each other about its circumference. The main body 110 of the shield 108 may include a plurality of resilient tabs 116 extending radially outwardly from the outer surface of the main body 110, which tabs 116 are also spaced apart from one another the same distance that the slots 114 are spaced apart on the circumference of the morcellator outer sleeve 104 so that the tabs 116 may be aligned with and lockingly received by the outer sleeve slots 114 to secure the shield 108 in place on the distal end of the outer sleeve 104 of the morcellator. Thus, the shield 108 may be mounted to the distal end of the outer sleeve 104 with a portion of the main body 110 of the shield being received within the axial bore of the outer sleeve, as shown in FIG. 3, for example. This particular mounting configuration for the shield 108 on the morcellator distal end is quite suitable and preferred, especially if the cutting blade is formed with a reduced diameter tip portion 115 so that the shield 108 will not interfere with the rotation of the cutting blade 100. Alternatively, the shield 108 may be formed with resilient tabs 114 extending radially outwardly from the inner surface of the main body 110 to resiliently snap into the slots 114 so that the shield is mounted on the outer surface of the outer sleeve 104. In such an embodiment, the tabs 116 would extend only so far into the slots 114 of the outer sleeve 104 as to securely mount the shield 108 on the distal end of the outer sleeve 104 but not so far as to interfere with the rotatable movement of the cutting blade 100.

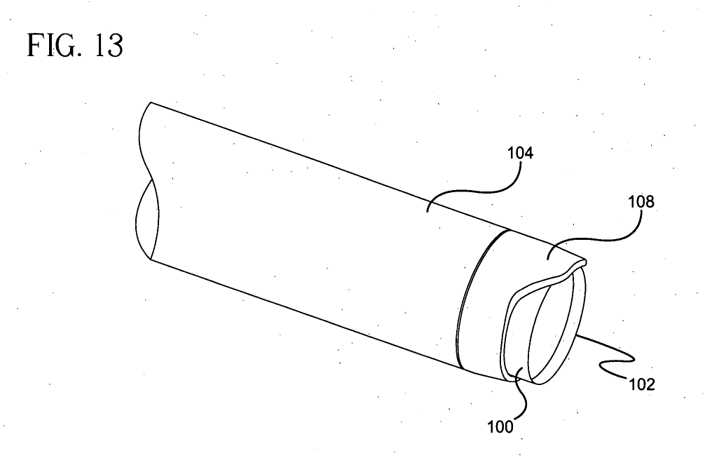

With the latter situation, where the shield 108 is integrally formed with the outer sleeve 104, the main body 110 of the shield 108 may be defined by the distal end portion of the cylindrical outer sleeve 104, with the protrusion 112 being defined by an axially extending portion of the outer sleeve 104, as shown in FIG. 13 of the drawings.

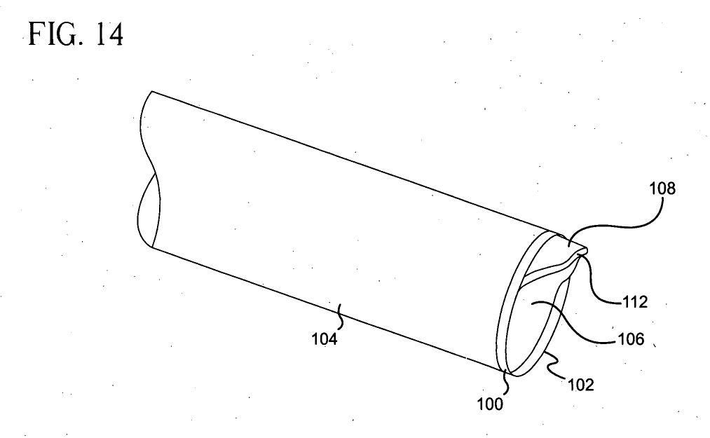

Also, it is envisioned to be within the scope of the present invention to form the inner sleeve 106 of the morcellator, if such is provided, with anti-coring structure. As shown in FIG. 14 of the drawings, the distal end portion of the inner sleeve 106 may define the cylindrical main body 110 of the shield 108, with the protrusion 112 of the shield 108 being defined by an axially extending portion of the inner sleeve 106. In such a situation, the outer sleeve 104 may be extended or retracted axially over the rotatable cutting blade 100 and the inner sleeve 106 to fully cover the sharpened edge 102 of the cutting blade 100 and to expose the sharpened edge 102, respectively, with the axially extending portion of the inner sleeve 106 extending beyond the sharpened edge 102 of the cutting blade 100 to promote efficient “orange peeling” and to prevent organ coring during a surgical procedure. Alternatively, or in combination with the axial movement of the outer sleeve 104, the inner sleeve 106 may move axially with respect to the rotatable cutting blade 100, such as in the manner described in the aforementioned Savage et al. patent, wherein the protrusion 112 extends beyond the periphery of the sharpened edge 102 of the cutting blade 100 or is retracted to a position where it does not protrude beyond the sharpened edge 102, such as when the “orange peeling” technique is not used in the surgical procedure. The mechanism to move either the outer sleeve 104 or the inner sleeve 106 of the morcellator to effect the desired positioning of the shield 108 with respect to the cutting blade 100 may be similar to or the same as the structure disclosed in the Savage, et al. patent.

When operating a surgical morcellator outfitted with the anti-coring device of the present invention, the surgeon would position the shield 108 of the anti-coring device in its first position in which the shield 108 covers the entire circumference of the sharpened edge 102 of the cutting blade 100. The surgeon would then insert the distal end of the outer sleeve 104 of the surgical morcellator through a small incision and into the patient's body cavity either with or without using a trocar. When performing an “orange peeling” technique in removing an organ, the shield 108 of the anti-coring device on the morcellator is positioned in the second position, as shown in FIG. 15 of the drawings, in which a portion of the sharpened edge 102 of the cutting blade 100 is covered by the protrusion 112 of the shield 108 and the remaining portion of the sharpened edge 102 is exposed. If the surgeon wishes to “core” the organ or tissue, he or she would retract the shield 108 axially on the cutting blade 100 to the third position in order to expose the entire circumference of the sharpened edge 102.

As can be seen from the foregoing description, the anti-coring device of the present invention can be positioned to cover only portions of the sharpened edge 102 of the cutting blade 100 and act as a “tool guide” to allow the maximum size tissue strip to be removed from the organ in an “orange peeling” surgical procedure by having the exposed sharpened edge 102 riding along the organ's outside surface, thus keeping the maximum amount of cutting edge diameter engaged with the organ's surface at all times. A surgical morcellator having such an anti-coring device requires less skill on the part of the surgeon while delivering the maximum tissue volume through the morcellator and, therefore, requiring less surgical time to complete the morcellation procedure. The anti-coring device of the present invention also enhances safety. Since the cutting blade 100 will not “core” into the organ, the blade 100 can be constantly seen by the surgeon through an endoscope, and the blade 100 location in the body cavity with respect to the organ being morcellated will always be observed. Furthermore, the tissue removed through the morcellation process may be stronger due to its larger cross-section, and longer strips of tissue may be withdrawn without breakage. If light pressure is maintained on the morcellator cutting blade 100 to force it partially into the organ being morcellated or an angle to the surface of the organ while pulling the transected tissue through the axial bore of the inner sleeve 106 or cutting blade 100, the tissue being morcellated is under slight compression due to the action of the anti-coring device. This leads to an even greater tissue volume removed by the cutting blade 100 of the morcellator and a quicker and more efficient morcellation procedure.

As is further evident from the foregoing description, the anti-coring device of the present invention may be suitably used with other forms of cutting elements, which broadly include the rotatable, sharpened edge cutting blade described previously, but also electrosurgical cutting devices, such as an electrosurgical coil through which is selectively passed an electric current. The anti-coring device of the present invention would be positioned to selectively cover and uncover an arcuate portion, or the entire circumference, of the electrosurgical coil, in a similar manner and operating in a similar way to that described previously with the rotatable cutting blade, the electrosurgical coil essentially replacing the sharpened cutting blade of the morcellator, with transected tissue morsels passing through the central opening of the electrosurgical coil.

Although illustrative embodiments of the present invention have been described herein with reference to the accompanying drawings, it is to be understood that the invention is not limited to those precise embodiments, and that various other changes and modifications may be effected therein by one skilled in the art without departing from the scope or spirit of the invention.

What is claimed is:

1. An anti-coring device for a surgical morcellator, the surgical morcellator including a rotatable cylindrical cutting blade having a distal end and a sharpened edge situated at the distal end, the anti-coring device comprising:
a shield situated at the distal end of the cutting blade and axially moveable with respect thereto, the shield including a main body having a bore formed axially therethrough for receiving a portion of the cutting blade, and a protrusion extending axially from the main body and partially about the circumference of the cutting blade, the shield being axially positionable on the cutting blade in a first position in which the main body thereof is disposed axially in alignment with the sharpened edge of the rotatable cutting blade to substantially cover the entire circumference of the sharpened edge of the cutting blade, and at least a second position in which the protrusion is disposed axially in alignment with the sharpened edge of the rotatable cutting blade to cover a selected arcuate first portion of the circumference thereof and to expose and not cover a second portion of the circumference of the sharpened edge of the rotatable cutting blade.

2. An anti-coring device for a surgical morcellator as defined by claim 1, wherein the surgical morcellator further includes an outer sleeve having a bore formed axially therethrough for receiving at least a portion of the rotatable cutting blade, the outer sleeve having a distal end situated in proximity to the distal end of the cutting blade, the outer sleeve being axially moveable on the rotatable cutting blade; and wherein the shield is situated on the distal end of the outer sleeve and axially moveable therewith to selectively cover and at least partially uncover the sharpened edge of the rotatable cutting blade.

3. An anti-coring device for a surgical morcellator as defined by claim 1, wherein the selected arcuate first portion of the circumference of the sharpened edge of the rotatable cutting blade covered by the protrusion is between about ninety degrees (90°) and about one hundred twenty degrees (120°) when the shield is in the at least second position.

4. An anti-coring device for a surgical morcellator as defined by claim 1, wherein the selected arcuate first portion of the circumference of the sharpened edge of the rotatable cutting blade covered by the protrusion is at least about fifty-four degrees (54°) when the shield is in the at least second position.

5. An anti-coring device for a surgical morcellator as defined by claim 1, wherein at least a portion of the protrusion of the shield extends axially beyond the sharpened edge of the rotatable cutting blade a first distance when the shield is in the at least second position.

6. An anti-coring device for a surgical morcellator as defined by claim 5, wherein the first distance which the portion of the protrusion extends axially beyond the sharpened edge of the rotatable cutting blade is between about 0.070 inches and about 0.100 inches.

7. An anti-coring device for a surgical morcellator as defined by claim 5, wherein the first distance which the portion of the protrusion extends axially beyond the sharpened edge of the rotatable cutting blade is at least about 0.030 inches.

8. An anti-coring device for a surgical morcellator as defined by claim 1, wherein the shield is axially positionable on the rotatable cutting blade in a third position in which the shield is in non-alignment with the sharpened edge of the rotatable cutting blade to expose and not cover the entire circumference of the sharpened edge.

9. A surgical morcellator having an anti-coring device attached thereto, the surgical morcellator comprising a rotatable cylindrical cutting blade having a distal end and a sharpened edge situated at the distal end, an outer sleeve having a bore formed axially therethrough for receiving at least a portion of the rotatable cutting blade, the outer sleeve having a distal end 5 situated in proximity to the distal end of the cutting blade, the outer sleeve being axially moveable on the rotatable cutting blade, the anti-coring device comprising a shield situated on the distal end of the outer sleeve and axially moveable therewith to selectively cover and at least partially uncover the sharpened edge of the rotatable cutting blade, the shield including a main body having a bore formed axially therethrough for receiving a portion of the cutting blade, and 10 a protrusion extending axially from the main body and partially about the circumference of the cutting blade, the shield being axially positionable on the rotatable cutting blade in a first position in which the main body thereof is disposed axially in alignment with the sharpened edge of the rotatable cutting blade to cover the entire circumference of the sharpened edge of the cutting blade, and at least a second position in which the protrusion is disposed axially in alignment with the sharpened edge of the rotatable cutting blade to cover a selected arcuate first portion of the circumference thereof and to expose and not cover a second portion of the circumference of the sharpened edge of the rotatable cutting blade.

10. A method of laparoscopically transecting tissue from an anatomical body of a patient during a surgical procedure, which comprises the steps of:
using a surgical morcellator having an anti-coring device, the surgical morcellator including a rotatable cylindrical cutting blade having a distal end and a sharpened edge situated at the distal end, and further including an outer sleeve having a bore formed axially therethrough for receiving at least a portion of the rotatable cutting blade, the outer sleeve having a distal end situated in proximity to the distal end of the cutting blade, the outer sleeve being axially moveable on the rotatable cutting blade, the anti-coring device including a shield situated on the distal end of the outer sleeve and axially moveable therewith to selectively cover and at least partially uncover the sharpened edge of the rotatable cutting blade, the shield including a main body having a bore formed axially therethrough for receiving a portion of the cutting blade, and a protrusion extending axially from the main body and partially about the circumference of the cutting blade, the shield being axially positionable on the cutting blade in a first position in which the main body thereof is disposed axially in alignment with the sharpened edge of the rotatable cutting blade to substantially cover the entire circumference of the sharpened edge of the cutting blade, and at least a second position in which the protrusion is disposed axially in alignment with the sharpened edge of the rotatable cutting blade to cover a selected arcuate first portion of the circumference thereof and to expose and not cover a second portion of the circumference of the sharpened edge of the rotatable cutting blade;
positioning the shield of the anti-coring device in the first position in which the shield substantially covers the entire circumference of the sharpened edge of the rotatable cutting blade;
inserting the distal end of the outer sleeve of the surgical morcellator into a patient;
positioning the shield of the anti-coring device in the at least second position in which at least the second portion of the circumference of the sharpened edge of the rotatable cutting blade is exposed; and
engaging the second portion of the sharpened edge of the morcellator cutting blade exposed by the shield when the shield is in the at least second position with the anatomical body of the patient for transecting tissue from the anatomical body.

11. An anti-coring device for a surgical morcellator, the surgical morcellator including a cutting element, the cutting element having a circumference associated therewith, the anti-coring device comprising:
a shield situated at the cutting element and axially moveable with respect thereto, the shield including a main body having a bore formed axially therethrough for receiving a portion of the cutting element, and a protrusion extending axially from the main body and partially about the circumference of the cutting element, the shield being axially positionable on the cutting element in a first position in which the main body thereof is disposed axially in alignment with the cutting element to substantially cover the entire circumference of the cutting element, and at least a second position in which the protrusion is disposed axially in alignment with the cutting element to cover a selected arcuate first portion of the circumference thereof and to expose and not cover a second portion of the cutting element.
